(19) United States
(12) Patent Application Publication
(10) Pub. No.: US 2008/0137813 A1
(43) Pub. Date: Jun. 12, 2008

(54) SUBJECT SUPPORT DEVICE AND X-RAY IMAGING APPARATUS

(76) Inventor: Hiroshi Umeda, Tokyo (JP)

Correspondence Address: Patrick W. Rasche;ARMSTRONG TEASDALE LLP, Suite 2600, One Metropolitan Square, St. Louis, MO, 63102-2740, US

(21) Appl. No.: 11/952,719

(22) Filed: Dec. 7, 2007

(30) Foreign Application Priority Data
Dec. 8, 2006 (JP) 2006-332160

(51) Int. Cl. A61B 6/04

(52) U.S. Cl. 378/208

(57) ABSTRACT

This invention provides a subject support device with a highly maneuverable top plate and an X-ray imaging apparatus with such subject support device. A subject support device comprises a top plate for a subject to lie on, and a pedestal base which cantilevers the top plate in a horizontally extendable manner, and a levelness maintaining unit which adjusts the posture of the pedestal base to maintain the levelness of a part for supporting the top plate. The levelness maintaining unit includes a detector which detects the levelness of the part for supporting the top plate, a control circuit which outputs a control signal according to a detection signal from the detector, and an actuator which changes the posture of the pedestal base according to a control signal from the control circuit.

### BACKGROUND OF THE INVENTION

The present invention relates to a subject support device and an X-ray imaging apparatus and more particularly to a subject support device having a top plate which is horizontally extended from a pedestal base, and the X-ray imaging apparatus having such subject support device.

An X-ray imaging apparatus employs a subject support device which supports a subject lying on it. One example of a subject support device is that a top plate is cantilevered by a pedestal base in a way to be horizontally extendable with a subject lying on it (for example, see JP-A No. 2002-177263).

### SUMMARY OF THE INVENTION

A rotation moment due to the load acts on the extended top plate. This rotation moment works toward inclining the top plate. When the amount of extension is larger or the load is larger, the rotation moment is larger and accordingly the inclination of the top plate is larger. Therefore, when moving the top plate manually to adjust the photographing position, the top plate's resistance in the pulling direction and that in the pushing direction are very different and maneuverability worsens.

Therefore, the problem for the present invention is to realize a highly maneuverable subject support device and an X-ray imaging apparatus having such subject support device.

The invention to solve the problem is, according to one aspect thereof, a subject support device characterized by comprising a top plate for a subject to lie on, a pedestal base which has a top plate supporting plane to support the top plate by its upper surface and cantilevers the top plate in a horizontally extendable manner by the top plate supporting plane, and levelness maintaining unit which can adjust the levelness of the top plate supporting plane.

The invention to solve the problem is, according to one aspect thereof, is an X-ray imaging apparatus including a subject support device which supports a subject and a photographing unit which x-rays a subject supported by the subject support device, characterized in that the subject support device comprises a top plate for a subject to lie on, a pedestal base which has a top plate supporting plane to support the top plate by its upper surface and cantilevers the top plate in a horizontally extendable manner by the top plate supporting plane, and levelness maintaining unit which can adjust the levelness of the top plate supporting plane.

For the purpose of maintaining levelness properly, it is preferable that the levelness maintaining unit can be automatically adjusted so as to maintain the levelness of the top plate supporting plane.

For the purpose of maintaining levelness properly, it is preferable that the levelness maintaining unit has a detector which detects the levelness of the top plate supporting plane.

For the purpose of maintaining levelness properly, it is preferable that the levelness maintaining unit has a control circuit which outputs a control signal according to a detection signal from the detector and adjusts the levelness of the top plate supporting plane according to a control signal from the control circuit.

For the purpose of maintaining levelness properly, it is preferable that the levelness maintaining unit has an actuator in a leg section of the pedestal base, and the actuator changes the posture of the pedestal base by expanding or contracting the leg section of the pedestal base in an unbalanced manner.

For the purpose of maintaining levelness properly, it is preferable that the leg section has an elastic part and a non-elastic part.

For the purpose of maintaining levelness properly, it is preferable that the leg section has a nonparallel link.

According to the present invention, a subject support device comprises a top plate for a subject to lie on, and a pedestal base which has a top plate supporting plane to support the top plate by its upper surface and cantilevers the top plate in a horizontally extendable manner by the top plate supporting plane, and levelness maintaining unit which can adjust the levelness of the top plate supporting plane, so that a subject support device with a highly maneuverable top plate and an X-ray imaging apparatus having such subject support device can be realized.

### BRIEF DESCRIPTION OF THE DRAWINGS

FIG. 1 is a view schematically showing the structure of an X-ray imaging apparatus as one example of the best mode for carrying out the invention.

FIG. 2 is a view schematically showing the structure of a subject support device as one example of the best mode for carrying out the invention.

FIG. 3 is a view schematically showing the structure of a subject support device as one example of the best mode for carrying out the invention.

### DETAILED DESCRIPTION OF THE INVENTION

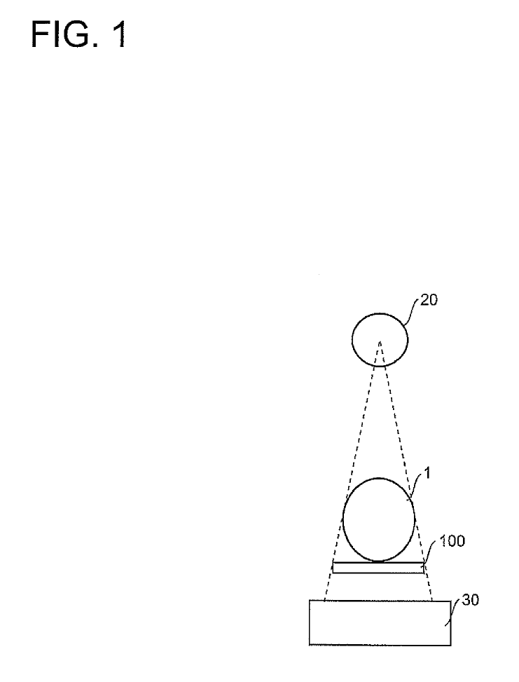

Next, the best mode for carrying out the invention will be described in detail referring to drawings. The present invention is not limited to the best mode for carrying out the invention. FIG. 1 shows a schematic structure of an X-ray imaging apparatus. The apparatus is one example of the best mode for carrying out the invention. The structure of the apparatus shows an example of the best mode for carrying out the invention concerning the X-ray imaging apparatus.

As shown in FIG. 1, the apparatus is designed so that a subject 1 supported by a subject support device 100 is X-rayed by an X-ray tube 20 and transmitted X rays are detected by an X ray sensor 30. It is also possible that the X ray sensor 30 is a photosensitive material which reacts to X rays.

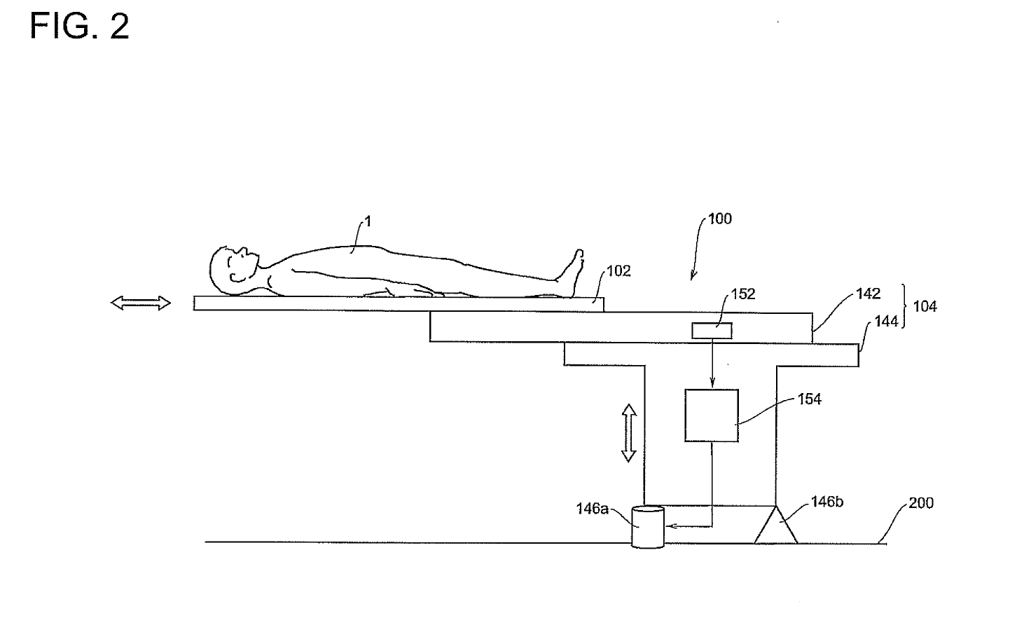

FIG. 2 schematically shows one example of the subject support device 100. The subject support device 100 is one example of the best mode for carrying out the invention. The structure of the subject support device 100 shows an example of the best mode for carrying out the invention concerning a subject support device.

As shown in FIG. 2, the subject support device 100 includes a top plate 102 for the subject 1 to lie on, a pedestal base 104 which cantilevers the top plate 102 in a horizontally extendable manner. The pedestal base 104 comprises an intermediate support part 142 and a base part 144 and the intermediate support part 142 supports the top plate 102 and the base part 144 supports the intermediate support part 142. Regarding the directions of horizontal movement of the top plate 102, the left hand in the figure is defined as the In direction and the right hand is defined as the Out direction.

The base part 144 stands upright on a floor 200 through legs 146a, 146b. The leg 146a is an elastic leg and the leg 146b is an non-elastic leg. The elastic leg 146a and the non-elastic leg 146b constitute an elastically unbalanced leg section. The elastic leg 146a is located on the In side and the non-elastic leg 146b is located on the Out side. The positions of the legs 146a and 146b may be changed.

The leg 146a is expanded or contracted by a built-in actuator. As the leg is expanded or contracted, the posture of the base part 144 changes. As the posture of the base part 144 changes, the levelness of the intermediate support part 142 to the ground changes.

The intermediate support part 142 has a detector 152 which detects the levelness. For example, a digital level or the like is used as the detector 152. Such a level is easily available in the market.

The base part 144 has a control circuit 154. A levelness detection signal is sent from the detector 152 to the control circuit 154. The control circuit 154 controls the amount of expansion/contraction of the leg 146a by a control signal according to the levelness detection signal. The amount of expansion/contraction of the leg 146a is controlled so that the intermediate support part 142 is level. The section consisting of the detector 152, control circuit 154 and leg 146a is one example of device for maintaining levelness in the present invention.

By such control, the intermediate support part 142 is always level regardless of the amount of extension of the top plate 102. Therefore, when a user manually moves the top plate 102 back and forth, there is no difference in resistance between movement toward the In direction and movement toward the Out direction and maneuverability is thus enhanced.

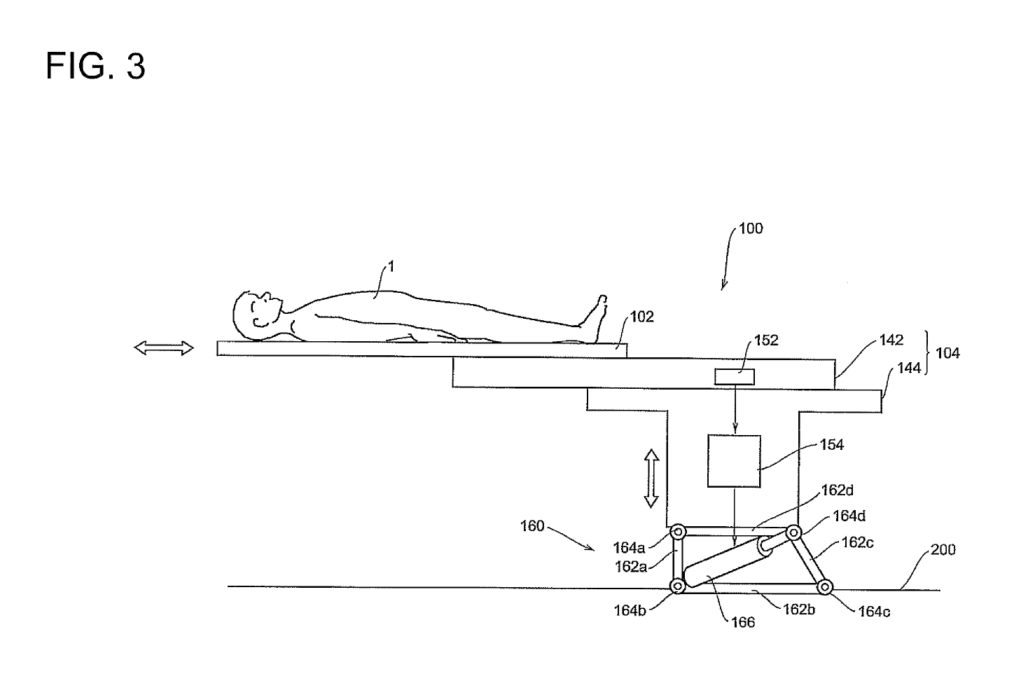

FIG. 3 schematically shows another example of the structure of the subject support device 100. The subject support device 100 is one example of the best mode for carrying out the invention. The structure of the subject support device 100 shows an example of the best mode for carrying out the invention concerning a subject support device.

In FIG. 3, the same elements as those shown in FIG. 2 are designated by the same reference numerals and their descriptions are omitted. As shown in FIG. 3, a leg section 160 of the base part 144 is structurally different from that shown in FIG. 2. The leg section 160 is a nonparallel link which includes four links 162a, 162b, 162c, 162d which are not equal in length and four joints 164a, 164b, 164c, 164d.

In this nonparallel link, a pair of joints 164b, 164d, diagonal to each other, are connected by an expandable shaft 166. The elastic shaft 166 incorporates an actuator. The actuator is driven according to a control signal from the control circuit 154.

As the shape of the nonparallel link changes with expansion or contraction of the expandable shaft 166, the leg section 160 expands or contracts in an unbalanced manner on the In side and Out side. As a consequence, the posture of the base part 144 changes and the levelness of the intermediate support part 142 changes and thus the control circuit 154 controls the amount of expansion/contraction of the expandable shaft 166 to maintain the levelness of the intermediate support part 142. The section consisting of the detector 152, control circuit 154 and leg section 160 is one example of device for maintaining levelness in the present invention.

1. A subject support device comprising:
a top plate for a subject to lie on;
a pedestal base which has a top plate supporting plane to support the top plate by its upper surface and cantilevers the top plate in a horizontally extendable manner by the top plate supporting plane; and
levelness maintaining unit which can adjust levelness of the top plate supporting plane.

2. The subject support device according to claim 1, wherein the levelness maintaining device can be automatically adjusted so as to maintain levelness of the top plate supporting plane.

3. The subject support device according to claim 1, wherein the levelness maintaining device has a detector which detects levelness of the top plate supporting plane.

4. The subject support device according to claim 3, wherein the levelness maintaining device has a control circuit which outputs a control signal according to a detection signal from the detector and adjusts levelness of the top plate supporting plane according to a control signal from the control circuit.

5. The subject support device according to claim 3, wherein the levelness maintaining device has a control circuit which outputs a control signal according to a detection signal from the detector and automatically adjusts levelness of the top plate supporting plane according to a control signal from the control circuit.

6. The subject support device according to claim 4, wherein the levelness maintaining device has an actuator in a leg section of the pedestal base and the actuator changes posture of the pedestal base by expanding or contracting the leg section of the pedestal base in an unbalanced manner.

7. The subject support device according to claim 5, wherein the levelness maintaining device has an actuator in a leg section of the pedestal base and the actuator changes posture of the pedestal base by expanding or contracting the leg section of the pedestal base in an unbalanced manner.

8. The subject support device according to claim 6, wherein the leg section has an elastic part and a non-elastic part.

9. The subject support device according to claim 7, wherein the leg section has an elastic part and a non-elastic part.

10. The subject support device according to claim 6, wherein the leg section has a nonparallel link.

11. An X-ray imaging apparatus comprising:
a subject support device which supports a subject; and
a photographing unit which x-rays a subject supported by the subject support device,
wherein
the subject support device comprises:
a top plate for a subject to lie on;
a pedestal base which has a top plate supporting plane to support the top plate by its upper surface and cantilevers the top plate in a horizontally extendable manner by the top plate supporting plane; and
levelness maintaining device which can adjust levelness of the top plate supporting plane.

12. The X-ray imaging apparatus according to claim 11, wherein the levelness maintaining device can be automatically adjusted so as to maintain levelness of the top plate supporting plane.

13. The X-ray imaging apparatus according to claim 11, wherein the levelness maintaining device has a detector which detects levelness of the top plate supporting plane.

14. The X-ray imaging apparatus according to claim 13, wherein the levelness maintaining unit has a control circuit which outputs a control signal according to a detection signal from the detector and adjusts levelness of the top plate supporting plane according to a control signal from the control circuit.

15. The X-ray imaging apparatus according to claim 13, wherein the levelness maintaining unit has a control circuit which outputs a control signal according to a detection signal from the detector and automatically adjusts levelness of the top plate supporting plane according to a control signal from the control circuit.

16. The X-ray imaging apparatus according to claim 14, wherein the levelness maintaining unit has an actuator in a leg section of the pedestal base and the actuator changes posture of the pedestal base by expanding or contracting the leg section of the pedestal base in an unbalanced manner.

17. The X-ray imaging apparatus according to claim 15, wherein the levelness maintaining unit has an actuator in a leg section of the pedestal base and the actuator changes posture of the pedestal base by expanding or contracting the leg section of the pedestal base in an unbalanced manner.

18. The X-ray imaging apparatus according to claim 16, wherein the leg section has an elastic part and a non-elastic part.

19. The X-ray imaging apparatus according to claim 17, wherein the leg section has an elastic part and a non-elastic part.

20. The X-ray imaging apparatus according to claim 16, wherein the leg section has a nonparallel link.
